(19) United States
(12) Patent Application Publication
(10) Pub. No.: US 2025/0025604 A1
(43) Pub. Date: Jan. 23, 2025

(54) 3D-PRINTABLE BIOCOMPOSITES

(71) Applicant: NewSouth Innovations Pty Limited, Sydney (AU)

(72) Inventors: Kiho CHO, Sydney (AU); Bellasanty Gangadhara PRUSTY, Sydney (AU); Ashish DIWAN, Sydney (AU)

(21) Appl. No.: 18/715,350

(22) PCT Filed: Dec. 1, 2022
(86) PCT No.: PCT/AU2022/051435

§ 371 Date: May 31, 2024

(30) Foreign Application Priority Data
Dec. 3, 2021 (AU) 2021903920

(51) Int. Cl. A61L 27/46; A61L 27/54; B33Y 10/00; B33Y 70/00; B33Y 80/00

(52) U.S. Cl. CPC A61L 27/46; A61L 27/54; B33Y 70/00; B33Y 80/00; A61L 2400/12; A61L 2400/18; A61L 2430/02; B33Y 10/00

(57) ABSTRACT

Disclosed herein are biocomposites which may be formed from compositions comprising: at least one monomer suitable for photopolymerisation; a photoinitiator, and a filler composition comprising at least one inorganic compound. The biocomposites may be formed in a process comprising 3D printing. Also disclosed herein are potential applications of the compositions and biocomposites, including in the optional treatment of bone defects or the repair of a portion of bone in a subject.

### CROSS-REFERENCE TO RELATED APPLICATIONS

The present application claims priority from Australian Provisional Patent Application No. 2021903920 filed on 3 Dec. 2021, the contents of which are incorporated herein by reference in their entirety.

### TECHNICAL FIELD

The present disclosure relates to biocomposites which may be formed from compositions comprising: at least one monomer suitable for photopolymerisation; a photoinitiator; and a filler composition comprising at least one inorganic compound. The biocomposites may be formed in a process comprising 3D printing. The biocomposites may also be used the treatment of bone defects or the repair of a portion of bone in a subject.

### BACKGROUND

The worldwide spinal implants and devices market was valued at USD9.5 B in 2020 and is expected to reach USD13.8 B by 2025 (Markets and Markets Research Private, 2020). By far, polyether ether ketone (PEEK) and titanium interbody fusion cages are both the most used as stand-alone devices in spinal fusion surgery. With the advanced 3D surgical imaging system, the introduction of 3D printed implants has been the emerging trend in the global spinal implant market and the spine field continues to expand applications for three-dimensional (3D) printing.

Among the global spine device companies, Orthofix has developed 3D printed titanium cervical interbody, the Construx Mini Ti Spacer System, which is designed with a functional gradient porous structure, and received FDA clearance in April 2021. Medtronic has launched a titanium 3D printed platform, TiONIC technology, which enables more complex designs and integrated surface technologies for spine surgery implants. This company also has introduced a Pure Titanium Coating (PTC™), on the PEEK implant surface, combining the benefits of both PEEK and titanium in a single interbody. Johnson & Johnson medical devices, Zimmer Biomet, Nexxt Spine and Stryker, have also developed 3D printed titanium implants. HAPPE Spine has patented a dense and porous dual design of hydroxyapatite (HAP) and PEEK composite materials through anatomy mimicking of cortical and cancellous bone.

The integration of functionalised materials and the optimised architectural structures is of great importance in developing orthopaedic implants such as interbody fusion cages (or spacers).

Titanium (or a titanium alloy) and PEEK are generally selected for medical implants due to their good mechanical properties and biocompatibility. Although conventional implants and spinal fusion cages provide a sufficient load-bearing capacity, their surfaces are generally inert or have limited ability to support osteointegration with the surrounding bone.

Consequently, a weaker interfacial bone-bonding can result in a higher implant failure causing bone instability, subsidence, implant migration, and severe pain or discomfort which may prompt a second surgery. Even though many surface coating techniques using osteoconductive/osteoinductive agents such as calcium phosphates on the implant structures have been developed to enhance bone/implant reactions, the potential risk of coating failure by fracture and delamination, which results from poor bonding between two different materials, can lead to a loosening of implants and bring a catastrophic failure of the implant.

The surfaces of titanium implants can stimulate bone growth. They are, however, not sufficient in some cases because they cause high radiodensity and magnetic field distortion at the tissue-metal interfaces on X-ray imaging, computed tomography, and magnetic resonance imaging. The induced secondary electrons from a metal surface by X-rays may contribute to DNA damage or DNA mutations near the metal implants.

The titanium and the PEEK cages are both associated with similar fusion rates, but the titanium cages (metal group), have led to an increased rate of subsidence, or the gradual penetration of the implant into the endplate surfaces, due to its high elastic modulus and stress shielding effect. On the other hand, PEEK cages (polymer group), are much more compliant compared to the titanium because their elastic modulus are nearly identical to the bones ranging between those of cortical and cancellous bones. However, PEEK is hydrophobic and biologically inert, which leads to low integration with surrounding tissues after implantation. Also, due to the semi-crystalline nature of PEEK and a high melting temperature, PEEK is highly susceptible to pre-/post-3D print processing conditions which leads to a large variation in the mechanical performance of PEEK structures. This has limited the adaption of PEEK 3D printing in medical applications where high-quality assurance and reproducibility are required.

To overcome these limitations raised from many conventional implants, new materials and/or methods are required.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each claim of this application.

### SUMMARY

Disclosed herein is a biocomposite formed from a composition comprising:
at least one monomer suitable for photopolymerisation
a photoinitiator and
a filler composition comprising at least one inorganic compound.

Also disclosed herein is a method of synthesising a biocomposite as disclosed herein, the method of comprising the steps of:
providing a composition comprising:
   at least one monomer suitable for photopolymerisation
   a photoinitiator and
   a filler composition comprising at least one inorganic compound and
3D printing the biocomposite.

An example schematic illustration of the proposed development process for multi-functionalised spinal fusion cages is shown in FIG. 1. In FIG. 1, several steps are exemplified: A—multi-functionalised resin system; B—3D printing; C—3D printed implant; D—3D printed spinal cage (hierarchical and porous); E—surface texturing; and F—smart fusion cage/implant.

Disclosed herein is a biocomposite formed from a method as disclosed herein.

Also disclosed herein is a method of treating or repairing a bone in a subject, for example a damaged or defective bone, the method comprising administering biocomposite as disclosed herein to a subject in need thereof.

Also disclosed herein is a biocomposite as defined herein for use in treating or repairing a bone in a subject, for example a damaged or defective bone.

Also disclosed herein is use of a composition comprising:
at least one monomer suitable for photopolymerisation
a photoinitiator and
a filler composition,
   in the formation of a biocomposite as disclosed herein.

Also disclosed herein is use of a biocomposite as disclosed herein in the formation of an article to treat or repair a bone in a subject.

The biocomposites disclosed herein may expand the application of orthopaedic implants/cages to ‘metal-free’ medical implants which require one or more of: good radiopacity, high mechanical strength, s and biological properties such as osteoconductivity, osteoinductivity, and/or biocompatibility. Exemplary collapse strength and compressive modulus of the biocomposites as defined herein and PEEK are shown in FIG. 4.

The presence of materials such as: nano-hydroxyapatites (n-HAPs), silica nano-particulates, and/or micro-sized short glass fibres in the composites could, amongst other possible advantages, increase the mechanical and/or biological performance of the materials in vivo.

Where the biocomposites are formed as 3D micro/nano-hierarchical porous structures of the implants (which may be fabricated through stereolithography (SLA) 3D printing and cold plasma surface treatment), bone growth and adhesion can be promoted. The 3D printed microporous structures in implants can potentially provide a greater contact area at the implant-new bone interface and long-term stability through bone ingrowth and mechanical interlocking. Also, the nanotextured surfaces of the implants by treatments such as cold argon-oxygen plasma treatment can produce multifunctional hierarchical topography with the surface-exposed n-HAPs that improve surfaces' hydrophilicity, osteointegration, and/or new bone development.

It will be appreciated that the embodiments of each aspect of the present disclosure may equally be applied to each other aspect, mutatis mutandis.

### BRIEF DESCRIPTION OF DRAWINGS

Whilst it will be appreciated that a variety of embodiments disclosed herein may be utilised, in the following, described herein are a number of examples with reference to the following drawings:

FIG. 1—Schematic illustration of the proposed development process for multi-functionalised spinal fusion cages.

FIG. 2—Exemplary implant, wherein: 1—body of a 3D printed multiscale-engineered orthopaedic implant; 2—multi-layered structure; and 3—micro porous structure.

FIG. 3—Images of nano hydroxyapatite.

FIG. 4—Collapse strength and compressive modulus of the biocomposites as described herein and PEEK.

FIG. 5—A surface nano-texturing on the biocomposites using atmospheric cold plasma etching process showing the change in hydrophilicity/hydrophobicity.

FIG. 6—A surface nano-texturing on the biocomposites using atmospheric cold plasma etching process.

FIG. 7—Surface etched thickness versus plasma etching time for n-HAP reinforced biocomposites.

### DETAILED DESCRIPTION

### General Definitions and Terms

With regards to the definitions provided herein, unless stated otherwise, or implicit from context, the defined terms and phrases include the provided meanings. In addition, unless explicitly stated otherwise, or apparent from context, the terms and phrases below do not exclude the meaning that the term or phrase has acquired by a person skilled in the relevant art. The definitions are provided to aid in describing particular embodiments, and are not intended to limit the claimed invention, because the scope of the invention is limited only by the claims.

The term “creeping substitution” will be understood to be referring to the process whereby a biocomposite graft or implant is gradually replaced or incorporated into a subject's body by living tissue. The subject's cells fill the porous surfaces of the biocomposite, and especially the inorganic structures comprised of such materials as hydroxyapatite, tricalcium phosphate, collagen, and others into new physical and physiological osteostructures, essentially converting the biocomposite or part of it into new bone.

All publications discussed and/or referenced herein are incorporated herein in their entirety, unless described otherwise.

Unless otherwise required by context, singular terms shall include pluralities and plural terms shall include the singular. Throughout this disclosure, unless specifically stated otherwise or the context requires otherwise, reference to a single step, composition of matter, group of steps or group of compositions of matter shall be taken to encompass one and a plurality (i.e., one or more) of those steps, compositions of matter, groups of steps or groups of compositions of matter. Thus, as used herein, the singular forms “a”, “an” and “the” include plural aspects unless the context clearly dictates otherwise. For example, reference to “a” includes a single as well as two or more; reference to “an” includes a single as well as two or more; reference to “the” includes a single as well as two or more and so forth.

Those skilled in the art will appreciate that the disclosure herein is susceptible to variations and modifications other than those specifically described. It is to be understood that the disclosure includes all such variations and modifications. The disclosure also includes all of the examples, steps, features, methods, compositions, formulations, and processes, referred to or indicated in this specification, individually or collectively, and any and all combinations or any two or more of said steps or features.

The term “and/or”, e.g., “X and/or Y” shall be understood to mean either “X and Y” or “X or Y” and shall be taken to provide explicit support for both meanings or for either meaning.

Unless otherwise indicated, the terms “first,” “second,” etc. are used herein merely as labels, and are not intended to impose ordinal, positional, or hierarchical requirements on the items to which these terms refer. Moreover, reference to a “second” item does not require or preclude the existence of lower-numbered item (e.g., a “first” item) and/or a higher-numbered item (e.g., a “third” item).

As used herein, the phrase “at least one of” or “one or more of” when used with a list of items, means different combinations of one or more of the listed items may be used and only one of the items in the list may be needed. The item may be a particular object, thing, or category. In other words, “at least one of” means any combination of items or number of items may be used from the list, but not all of the items in the list may be required. For example, “at least one of item A, item B, and item C” may mean item A; item A and item B; item B; item A, item B, and item C; or item B and item C. In some cases, “at least one of item A, item B, and item C” may mean, for example and without limitation, two of item A, one of item B, and ten of item C; four of item B and seven of item C; or some other suitable combination.

It is to be appreciated that certain features that are, for clarity, described herein in the context of separate embodiments, may also be provided in combination in a single embodiment. Conversely, various features that are, for brevity, described in the context of a single embodiment, may also be provided separately or in any sub-combination.

Throughout the present specification, various aspects and components of the disclosure can be presented in a range format. The range format is included for convenience and should not be interpreted as an inflexible limitation on the scope of the present disclosure. Accordingly, the description of a range should be considered to have specifically disclosed all the possible sub-ranges as well as individual numerical values within that range, unless specifically indicated. For example, description of a range such as from 1 to 5 should be considered to have specifically disclosed sub-ranges such as from 1 to 3, from 1 to 4, from 1 to 5, from 2 to 4, from 2 to 5, from 3 to 5 etc., as well as individual and partial numbers within the recited range, for example, 1, 2, 3, 4 and 4.5, unless where integers are required or implicit from context. This applies regardless of the breadth of the disclosed range. Where specific values are required, these will be indicated in the specification.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

Throughout this specification, the term “consisting essentially of” is intended to exclude elements which would materially affect the properties of the claimed composition, method or process.

The terms “comprising”, “comprise” and “comprises” herein are intended to be optionally substitutable with the terms “consisting essentially of”, “consist essentially of”, “consists essentially of”, “consisting of”, “consist of” and “consists of”, respectively, in every instance.

Herein, unless indicated otherwise, the term “about” encompasses a 10% tolerance in any value or values connected to the term.

Herein “weight %” may be abbreviated to as “wt %” or “wt. %”.

The recipients of the recited biocomposites are referred herein with the interchangeable terms “patient”, “recipient” “individual”, and “subject”. These four terms are used interchangeably and refer to any human or animal (unless indicated otherwise), as defined herein.

The recipients of biocomposite described herein, can be a human being, of any gender. Alternatively, the recipients of a biocomposite described herein, e.g., a patient or subject, can also be a non-human animal. “Non-human animals” or “non-human animal” is directed to the kingdom Animalia, excluding humans, and includes both vertebrates and invertebrates, male or female, and comprises: warm blooded animals, including mammals (comprising but not limited to primates, dogs, cats, cattle, pigs, sheep, goats, rats, guinea pigs, horses, or other bovine, ovine, equine, canine, feline, rodent or murine species), birds, insects, reptiles, fish and amphibians.

Herein the term “active agent” refers to any agent that is capable of providing a therapeutic, prophylactic or other biological effect within a subject or patient. An active agent can also be a diagnostic agent, or be for enhancing healing at an in vivo site. Unless specifically defined, or clear from context, the term “pharmaceutically active agent” or “active agent” is used herein can refer to any a protein, peptide, sugar, saccharide, nucleoside, inorganic compound, lipid, nucleic acid, small synthetic chemical compound, or organic compound that appreciably alters or affects the biological system to which it is introduced.

### Biocomposite

Disclosed herein is a biocomposite formed from a composition comprising:
at least one monomer suitable for photopolymerisation
a photoinitiator and
a filler composition comprising at least one inorganic compound.

Also disclosed herein is a method of synthesising a biocomposite as defined herein, the method of comprising the steps of:
providing a composition comprising:
   at least one monomer suitable for photopolymerisation
   a photoinitiator and
   a filler composition and
3D printing the biocomposite.

Also disclosed herein is a biocomposite formed from a method as described herein.

Also disclosed herein is use of a composition comprising:
at least one monomer suitable for photopolymerisation
a photoinitiator and
a filler composition,
   in the formation of a biocomposite as described herein.

In one embodiment the biocomposite is formed via 3D printing. In some embodiments, a composition used in the formation of a biocomposite is fluidic in its uncured state, allowing for 3D printing. Examples of appropriate 3-D printing methods include, but are not limited to: stereolithography (SLA), digital light processing (DLP), and masked stereolithography (MSLA). For 3D printing applications, rheological properties may be tailored in relation to: the specific application, the specific monomers being utilised and/or the presence of any additional additives.

In one embodiment the biocomposite is formed via 3D printing using a light source to initiate photopolymerisation of the materials or compositions used in the formation of the biocomposite. The light source may be an ultraviolet (UV) light source. The light source may have a wavelength in the range of about 280 nm to about 400 nm. For example a wavelength of about, or at least about: 280 nm, 285 nm, 290 nm, 295 nm, 300 nm, 305 nm, 310 nm, 315 nm, 320 nm, 325 nm, 330 nm, 335 nm, 340 nm, 345 nm, 350 nm, 355 nm, 360 nm, 365 nm, 370 nm, 375 nm, 380 nm, 385 nm, 390 nm, 395 nm, or 400 nm.

In some embodiments the biocomposite is photocured during the hardening process. In some embodiments the photocuring is performed with UV light. In some embodiments the photocuring is performed with UV light. In some embodiments the photocuring process also sterilises the biocomposite.

In one embodiment, the biocomposite may be adapted to tailor it to a specific application and/or specific subject.

In one embodiment, a layer comprising one or more components is applied to at least a portion of a surface of the biocomposite. In some embodiments, the layer comprising one or more component comprises hydroxyapatite, and glass fibres and/or glass particles. For example, wherein a layer comprising hydroxyapatite is applied to at least a portion of one or more surfaces of the biocomposite. In one embodiment, this layer provides an increase in the bioactivity and mechanical properties of the surface layer. This layer may be applied in a technique known in the art and as disclosed herein. For example, one or more of the layers may be applied using a 3D printing technique.

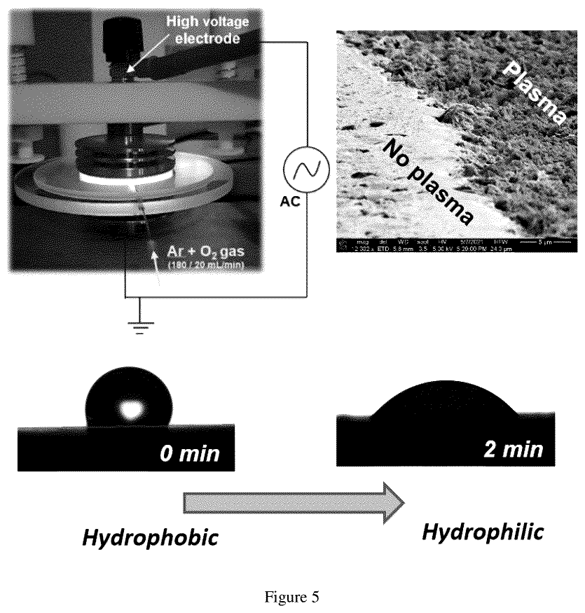

The biocomposite may be used directly once formed from the composition. Alternatively, at least one surface of the biocomposite may be modified in a process, optionally selected from: chemical etching, chemical coating (optionally chemical grafting), electrochemical grafting, laser etching, mechanical surface modifications, plasma-assisted coating, plasma-assisted etching, physical vapour deposition (optionally with a plasma or ion beam), chemical vapour deposition, atomic layer deposition, or mixtures thereof. FIGS. 5 to 7 display the following: a surface nano-texturing on the biocomposites using atmospheric cold plasma etching process showing the change in hydrophilicity/hydrophobicity with water droplets; a surface nano-texturing on the biocomposites using atmospheric cold plasma etching process; and surface etched thickness versus plasma etching time for n-HAP reinforced biocomposites.

In one embodiment, at least one surface is etched to remove at least a portion of said surface, wherein the etching comprises at least one of: chemical etching, laser etching, plasma-assisted etching or mechanical etching. Any etching or modification to the biocomposite may be undertaken at a temperature below the glass transition temperature (T_{g}), of one or more monomers or polymers used in the formation of the biocomposite. In one embodiment, wherein the etching (or another process as disclosed herein, is undertaken at a temperature in a range of about 20° C. to about 50° C., optionally in a range of about 25° C. to about 42° C. For example, a temperature of about, or at least about: 20° C., 25° C., 30° C., 35° C., 40° C., 45° C., or 50° C.

Herein, at least one surface of the biocomposite may be treated with a plasma. For example, at least one surface may be etched with a plasma to remove at least a portion of said surface. Any appropriate plasma, known in the art, may be used for modifying the biocomposite, for example the plasma may be: an argon-oxygen plasma, an oxygen plasma, a helium plasma, a nitrogen plasma, an argon plasma, or combinations thereof. In one embodiment the plasma treatment is at a temperature in a range of about 20° C. to about 40° C. For example, a temperature of about, or at least about: 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., or 40° C.

Any modification of the biocomposite may change the chemical composition of at least one surface of the biocomposite. For example, treatment with a plasma may increase the concentration of at least one of: hydroxyl groups, hydroxyl radicals and/or or reactive oxygen species, on at least a portion of the surface treated with the plasma. At least one surface of the biocomposite may be antimicrobial or display antimicrobial properties, for example following a treatment such as a plasma treatment.

The biocomposite may be formulated to be visualised in vivo using low dose X-rays. In one embodiment, the low dose X-rays is between about 52 kV×1.9 mAs to about 60 kV×6.2 mAs.

### Monomers/Polymers

A composition used in the formation of a biocomposite preferably comprises at least one monomer suitable for photopolymerisation.

In one embodiment the biocomposites are formed via photopolymerisation, wherein a light source is used light (for example visible or UV light) to initiate and/or propagate a polymerisation reaction. In another embodiment the photopolymerisation is a radical polymerisation.

In one embodiment at least one monomer comprises a functional group that is capable of being utilised in a radical polymerisation. At least one monomer suitable for photopolymerisation may comprise at least one photopolymerisable group selected from, but not limited to: alkene, allyl, vinyl, methacrylate and/or acrylate. In one embodiment at least one monomer comprises at least alkene group. In one embodiment at least one monomer comprises at least one acrylate group. In one embodiment at least one monomer comprises at least one methacrylate group. In one embodiment at least one monomer comprises at least one vinyl group. In one embodiment at least one monomer comprises at least one allyl group.

At least one monomer suitable for photopolymerisation may be selected from, but not limited to: bisphenol A glycidyl methacrylate (Bis-GMA), ethoxylated bisphenol A dimethacrylate (Bis-EMA), urethane dimethacrylate (UDMA), triethylene glycol dimethacrylate (TEGDMA), decanediol dimethacrylate (D₃MA), 2-hydroxyethyl methacrylate (HEMA), and mixture thereof. In some embodiments, the monomer suitable for photopolymerisation may be selected from, but not limited to: mono-(meth)acrylate, di(meth)acrylate, tri-(meth)acrylate) or dimethacrylate monomers (e.g. PEGMA, UDMA, HDDMA and TEGDMA. In some embodiments, a mixture of UDMA and TEGDMA can be used. In some embodiments, the ratio of the mixture of UDMA and TEGDMA is 8:2.

At least one monomer suitable for photopolymerisation may comprise at least one monomer comprising a plurality of photopolymerisable groups, optionally selected from: alkene, ally, vinyl, methacrylate and/or acrylate groups. At least one monomer suitable for photopolymerisation may comprise: two or three methacrylate groups; two or three acrylate groups; two or three vinyl groups; or two or three allyl groups.

In some embodiments at least one monomer may be used as a “base”, for example one or more of Bis-GMA, Bis-EMA and/or UDMA. In some embodiments, at least one monomer may be a diluent or co-monomer for one or more base monomers, for example TEGDMA, D₃MA, and/or HEMA.

The photopolymerisable monomers may be set or hardened (cured) by the methods known in the art. Common methods include, for example, exposure to light (e.g. at 400-470 nm), oxidation by exposure to air, oxidation by exposure to a chemical oxidant present in the composition (usually mixed into the biocomposite immediately prior to use) or other forms of chemical reactions that initiate polymerisation of the monomer.

In one embodiment the biocomposite is crosslinked. The polymers formed from the composition comprising at least one photopolymerisable monomer may react to form interconnecting linkages between polymer chains (or as growth sites for the copolymer chains), thereby crosslinking the final biocomposite. In an alternative embodiment, the biocomposite may not be crosslinked, but optionally be capable of crosslinking.

One potential advantage obtained from crosslinking is the stability of the resulting biocomposite. For example crosslinking can reduce the solubility of the biocomposite in comparison to similar compositions which are not crosslinked. In addition, the crosslinked nature of the biocomposite may increase the chemical and/or biological resistance of the biocomposite. The use of crosslinking may also yield a biocomposite with a surface which has an increased heat tolerance, decreased permeability, better abrasion resistance and/or extend the life, in comparison to biocomposites which are not crosslinked. Crosslinking may also increase desirable mechanical properties. Crosslinking may occur due to hydrogen bonding. Hydrogen bonding can be either intramolecular bonds between moieties included in segments of a polymer, or inter-molecular between one or more polymers. Physical crosslinking may be achieved by specific hydrogen bonding between polymer segments. Crosslinking may be introduced via the use of reagents or polymer segments that are branched and comprise a plurality of branches or arms.

The acidic nature of the degradation products of common biodegradable materials, such as polylactic acid (PLA) and polyglycolic acid (PGA) may lead to unpredictable clinical results including scarring and narrowing at the interface with native tissue. In one embodiment, the biocomposites may be formulated to allow for creeping substitution, as opposed to formulating the biocomposites to degrade in vivo and/or in vitro.

One or more monomers used in the formation may be functionalised with a functional moiety selected from, but not limited to: a hydroxyl group, an amine group, a thiol group, a carboxylic group, a carbonyl group, a halo group, a nitro group, and mixtures thereof.

The one or more monomers may be present in a range of about: 70 to about 95 wt % of the biocomposite. For example, the one or more monomers are present in an amount of about, or at least about: 70 wt %, 72.5 wt %, 75 wt %, 77.5 wt %, 80 wt %, 82.5 wt %, 85 wt %, 87.5 wt %, 90 wt %, 92.5 wt %, or 95 wt %, of the biocomposite.

### Photoinitiator

A composition used in the formation of a biocomposite as disclosed herein may comprise at least one photoinitiator.

Examples of photoinitiators include, but are not limited to: camphorquinone (CQ), bisacylphosphine oxide (BAPO), benzophenone (BP), N, N-dimethyl-p-toluidine (DMPT), ethyl-4-(dimethylamino)benzoate (EDMAB), and 2-4-6-trimethylbenzoyl-diphenyl-phosphine oxide (TPO), and mixtures thereof.

The concentration of one or more photoinitiators will be dependent on a number of factors including, but not limited to: the type of photoinitiator, the monomers used, the concentration of the monomers, and/or the conditions (for example wavelength of light) utilised to produce the biocomposite.

### Filler Composition

A composition used in the formation of a biocomposite preferably comprises a filler composition.

The filler composition may comprise one or more inorganic compounds. The filler composition may comprise at least one compound selected from: hydroxyapatite, silica, for example silica particulates, glass powder, glass fibres (optionally selected from E type and S type glass type fibres), and mixtures thereof.

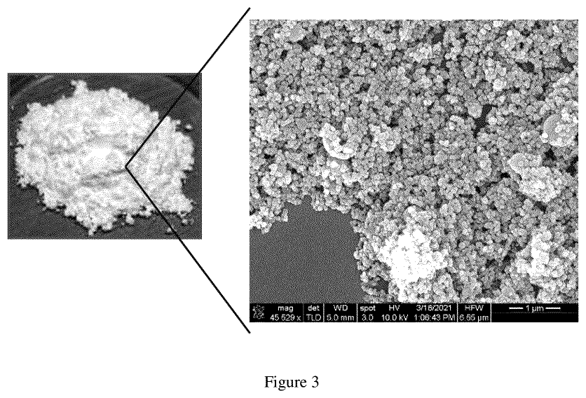

In one embodiment the filler composition comprises hydroxyapatite, optionally in a particulate form. Images of nano hydroxyapatite are shown in FIG. 3. The particulate form may be selected from nano-spheres, nano-whisker, and/or nano-rods. The hydroxyapatite, optionally in a particulate form, may have a diameter of about 50 nm to about 200 nm. For example the diameter may be about, or at least about: 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, 150 nm, 155 nm, 160 nm, 165 nm, 170 nm, 175 nm, 180 nm, 185 nm, 190 nm, 195 nm, or 200 nm.

The filler composition may comprise silica nano-particulates, optionally having a particle size of about 50 nm to about 700 nm. For example, the diameter may be about, or at least about: 50 nm, 100 nm, 150 nm, 200 nm, 250 nm, 300 nm, 350 nm, 400 nm, 450 nm, 500 nm, 550 nm, 600 nm, 650 nm, or 700 nm.

In some embodiments the filler composition as described herein, may be any fibres or fibrous material such as polymer fibres or glass fibres. The filler composition may comprise glass fibres (optionally selected from E type and S type). In some embodiments, the filler composition may comprise bioactive glass fibre. In some embodiments, the filler composition may comprise high strength fibres, being fibres having a tensile strength of above about 1 GPa (as measured by ASTM C1557-14) and/or a flexural strength of greater than about 150 MPa (using ISO 4049 three point flexural testing).

In an embodiment, the glass fibres may be selected from aluminosilicate glass, barium glass, fluorine glass, quartz, fused silica, borosilicate glass, aluminofluorosilicate glass, high calcium glass, high magnesium glass and mixtures thereof. In an embodiment, the glass is an S-glass (structural glass). S-glass is typically an alumino silicate glass having negligible CaO content and a high MgO content. S-glass is so named for “stiff glass”, due to its high tensile strength or modulus. Examples of other types of glass, glass fibres or fibreglass that may be used include E-glass (an aluminoborosilicate glass with less than 1% w/w alkali oxides), A-glass (an alkali lime glass with little or no boron oxide), E-CR glass (an electrical/chemical resistant alumino-lime silicate glass having less than about 1% w/w alkali oxides), C-glass (an alkali lime glass having high boron oxide content), D-glass (a borosilicate glass named for its low dielectric constant) and R-glass (alumino silicate glass having negligible MgO and CaO content used for high mechanical requirements as reinforcement). In one embodiment the filler comprises a glass powder. In another embodiment the filler comprises a mortar composition of glass powder and concrete. In another embodiment the mortar composition comprises about 10% to about 40% glass powder. In yet another embodiment the filler comprises one or more of: GP10, GP20, GP30 and/or GP40.

Glass fibres are commercially available in many different diameters. In one embodiment, obtaining glass fibres having the desired aspect ratio typically involves selecting glass fibre having the desired diameter and cutting the length accordingly.

The glass fibres may have a length of about 250 to about 350 μm. For example a length of about, or at least about: 250 μm, 260 μm, 270 μm, 280 μm, 290 μm, 300 μm, 310 μm, 320 μm, 330 μm, 340 μm, or 350 μm. The glass fibres may have a diameter of about 5-10 μm. For example, a diameter of about, or at least about: 5 μm, 6 μm, 7 μm, 8 μm, 9 μm, or 10 μm.

The filler composition may be present in a range of about 5 to about 30 wt % of the biocomposite. For example, in an amount of about, or at least about: 5 wt %, 7.5 wt %, 10 wt %, 12.5 wt %, 15 wt %, 17.5 wt %, 20 wt %, 22.5 wt %, 25 wt %, 27.5 wt %, or 30 wt %.

### Further Additives

At least one additional additive may be added prior, during or after the formation of the biocomposite. Examples of an additional additive may be selected from: plasticiser, dye, pigment, modifier, stabiliser, acid scavenger, compatibiliser, other polymers, or mixtures thereof. The at least one additive may be a pharmaceutically active compound.

Examples of suitable active agents include, but are not limited to, synthetic inorganic and organic compounds, drugs, proteins, peptides, polysaccharides and other sugars, lipids, and oligonucleotides, and DNA and RNA nucleic acid sequences, and the like. In one embodiment a patient's cells could be used as an active agent. Furthermore, peptides such as endothelial cells adhesive ligands and proteins, such as growth factors, could also be incorporated to a structure composed partially or wholly of a copolymer and/or composition as defined herein.

A biocomposite may comprise one or more growth factors. In a broad form, the one or more growth factors may be one or more substances that promote and/or regulate cell division and cell survival. For example, one or more growth factors may be selected from, but not limited to: one or more of Bone Morphogenic Proteins (BMP's) or their active fragments including BMP2, BMP4, BMP6, BMP7, BMP9, BMP 14 etc.; platelet derived growth factors (PDGF), e.g., PDGF AA, PDGF BB; insulin-like growth factors (IGF), e.g., IGF-I, IGF-II; fibroblast growth factors (FGF), e.g., acidic FGF, basic FGF, β-endothelial cell growth factor, FGF 4, FGF 5, FGF 6, FGF 7, FGF 8, and FGF 9; transforming growth factors (TGF), e.g., TGF-β1, TGF-β1.2, TGF-β2, TGF-β3, TGF-β5; vascular endothelial growth factors (VEGF), e.g., VEGF, epidermal growth factors (EGF), e.g., EGF, amphiregulin. betacellulin, heparin binding EGF; interleukins, e.g., IL-1, IL-2, IL-3, IL-4. IL-5, IL-6, IL-7, IL-8, IL9, IL-10, IL-11, IL-12, IL-13, IL-14; colony stimulating factors (CSF). e.g., CSF-G, CSF-GM, CSF-M, BMP cytokine proteins; nerve growth factor (NGF); stem cell factor; hepatocyte growth factor, and ciliary neurotrophic factor.

Polyols, such as glycerol or sorbitol, may be used as plasticisers, for example to facilitate 3D printing.

Exemplary plasticisers include, but are not limited to: materials such as mineral oils, low molecular weight esters, glycol ethers, glycol ether esters of aliphatic acid, glycol ether esters of aliphatic diacid, glycol ether esters of cinnamic acid, polyethylene glycol, polypropylene glycol, ortho and terephthalates, citrates, adipates, combinations, mixtures, and the like. In a particular embodiment, the plasticizer includes a glycol ether, or esters of glycol ether with aliphatic acids or aliphatic diacides, or esters of glycol ether with cinnamic acid, diethylene glycol dibutyl ether, bis[2-(2-butoxyethoxy)ethyl]adipate, bis(2-butoxyethyl)sebacate, bis[2-(2-butoxypropoxy)propyl]adipate, bis[2-(2-butoxypropoxy)propyl]sebacate, bis(2-ethoxyethyl)adipate, bis(2-ethoxyethyl)sebacate, dipropylene glycol methyl ether acetate, dipropylene glycol methyl ether cinnamate, diethyl-ene glycol butyl ether cinnamate, dipropylene glycol butyl ether acetate, tripropylene glycol methyl ether acetate, tripropylene glycol methyl ether butyrate, tripropylene glycol butyl ether acetate, diethylene glycol methyl ether acetate, diethylene glycol butyl ether acetate, triethylene glycol butyl ether acetate, triethylene glycol methyl ether acetate, combinations thereof, and the like. In a particular embodiment, the plasticiser may be used to lower the T_{g} of the resulting blend, enabling the blend to be within the elastomeric region and flexible at room temperature (i.e. about 25° C.). The plasticizer may be present in an amount of about 1% to about 60%, such as about 2% to about 45%, by weight of the total weight of the composition.

Exemplary modifiers include, but are not limited to: any reasonable modifiers such as additional nucleating agents like boron nitride or crosslinking agents such as silanes or diisocyanates.

Exemplary stabilisers include, but are not limited to: any reasonable stabilisers such as hindered amines, phenolic UV stabilizers, metal based heat stabilizers, butylated hydroxytoluene, and combinations thereof.

Exemplary acid scavengers include, but are not limited to, any reasonable acid scavengers such as calcium or zinc stearates.

Exemplary compatibilisers include, but are not limited to, any reasonable compatibilisers such as low to medium molecular weight polymers acting similar to surfactants.

In a particular embodiment, additives may be used such as other polymers to lower the T_{g} of the resulting composition and/or final biocomposite.

The biocomposite may be substantially free or free of transition/heavy metal. The metal may optionally be selected from titanium, iron, aluminium, magnesium, and copper, and alloys thereof.

The biocomposite may be substantially free or free of a polyaryletherketone. The polyatyletherketone may be selected from: polyether ether ketone (PEEK), polyether ketone (PEK), polyether ether ketone (PEEKK), polyether ketone (PEKK), or a mixture thereof).

### Applications

Disclosed herein is a biocomposite for the treatment or repair of a bone in a subject.

Also disclosed herein is a method of treating or repairing bony defects, for example as a consequence of low energy or high energy trauma, tumour, infection, for example in the metaphysis, subchondral regions or diaphysis or any regions in the flat bones or vertebrae; in a subject, the method comprising administering a biocomposite disclosed herein to a subject in need thereof.

Also disclosed herein is use of a biocomposite as disclosed herein in the formation of an article to treat or repair damaged or defective bone in a subject.

The treatment or repair may be selected from the biocomposite being shaped in an appropriate form, such as a screw, plate, rod or moulded to fill a defect by pre made modular forms or by printing following measurement of the defect by CT scan dicom data or the like, enabling a personalised solution for a subjects specific bone related pathology.

The treatment or repair may involve one or more bones in a subject, for example the bone may be a broken shaft of radius or ulna that requires a plate with screws, a tibial shaft fracture that requires a intramedullary nail with interlocking screws, a plateau or plafond fracture of the proximal or distal tibia that may need bone graft augmentation with plates and screws, a low impact fracture of the vertebral body that require poly-methyl methacrylate injection, a fracture of the distal radius that requires a plate, screws with or without bone graft augmentation. In non-fracture situations the current biocomposites or compositions disclosed herein may be applied to situations like spinal fusion for degenerative disc disease and scoliosis corrections. Herein, the biocomposite can be designed as one of the many interbody devices including those for anterior, lateral, posterior, transforaminal and oblique interbody fusions, either in the lumbar, thoracic or cervical regions. Similarly in non-fracture conditions rods, screws and plates can be designed using the biocomposites as herein described.

Herein, the biocomposite may be formulated or manufactured in as an article, and the article may be a medical device. Examples of medical devices include bone replacement endo prosthetic devices; bone fixation devices like screws and plates and intramedullary rods; bone augmentation devices like cancellous bone fillers for bony voids and defects.

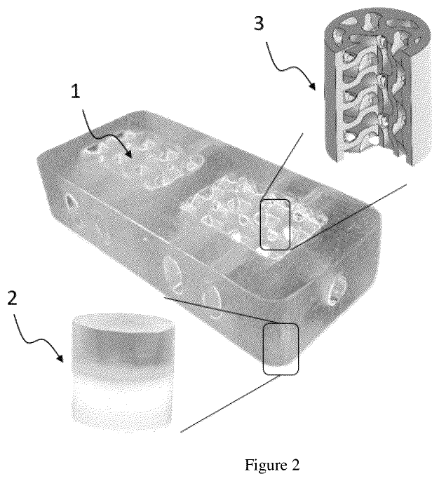

Herein, the biocomposite and/or article may be in the form of an implant, such implants may include being a part component of a joint replacement prosthesis like the stem of a femoral component of a hip arthroplasty device, or part of the tibial or femoral component of a knee arthroplasty device and similarly the said bio-composites may be utilised in making parts of other joint replacements like shoulders, elbows, wrists, intervertebral discs, ankles etc. An exemplary implant is shown in FIG. 2.

Herein, the biocomposite and/or article may be in the form of an orthopaedic implant.

Herein, the biocomposite and/or article may be in the form of an orthopaedic screw, rod, or plate.

### Imaging

Disclosed herein is a biocomposite that can be imaged with low dosage x-ray, in a subject. In one embodiment, the low dosage X-rays is between about 52 kV×1.9 mAs to about 60 kV×6.2 mAs

The low dosage X-ray is not highly ionizing and may reduce the likelihood of DNA damage in a subject.

### EXAMPLE EMOBIDMENTS

The present disclosure may be described by one or more of the following example embodiments:
1. A biocomposite formed from a composition comprising:
   at least one monomer suitable for photopolymerisation
   a photoinitiator and
   a filler composition comprising at least one inorganic compound.
2. The biocomposite according to example embodiment 1, wherein the biocomposite is formed via 3D printing, optionally stereolithography 3D printing, masked stereolithography 3D printing, or digital light processing.
3. The biocomposite according to example embodiment 1 or example embodiment 2, wherein the biocomposite is formed via 3D printing using a ultraviolet light source.
4. The biocomposite according to example embodiment any one of example embodiments 1 to 3, wherein the biocomposite is formed via 3D printing using a light source with a wavelength in the range of about 280 nm to about 400 nm.
5. The biocomposite according to any one of the preceding example embodiments, wherein:
   the biocomposite further comprises at least one of: hydroxyapatite, n-hydroxyapatite, glass fibres and/or glass particles or
   a layer comprising: hydroxyapatite, n-hydroxyapatite, glass fibres and/or glass particles is applied to at least a portion of one or more surfaces of the biocomposite.
6. The biocomposite according to example embodiment 5, wherein the layer comprising hydroxyapatite is applied using a 3D printing technique.
7. The biocomposite according to any one of the preceding example embodiments wherein at least one surface of the biocomposite is modified in a process selected from: chemical etching, chemical coating (optionally chemical grafting), electrochemical grafting, laser etching, mechanical surface modifications, plasma-assisted coating, plasma-assisted etching, physical vapour deposition (optionally with a plasma or ion beam), chemical vapour deposition, atomic layer deposition, or mixtures thereof.
8. The biocomposite according to any one of the preceding example embodiments, wherein at least one surface of the biocomposite is etched to remove at least a portion of said surface, wherein the etching optionally comprises at least one technique selected from: chemical etching, laser etching, plasma-assisted etching, mechanical etching, or mixtures thereof.
9. The biocomposite according to example embodiment 8, wherein the etching is undertaken at a temperature in a range of about 20° C. to about 50° C., optionally in a range of about 25° C. to about 42° C.
10. The biocomposite according to any one of the preceding example embodiments wherein at least one surface of the biocomposite is treated with a plasma.
11. The biocomposite according to example embodiment 10, wherein the treatment with a plasma increases the concentration of at least one of: hydroxyl groups, hydroxyl radicals and/or or reactive oxygen species, on at least a portion of the surface treated with the plasma.
12. The biocomposite according to any one of the preceding example embodiments wherein at least one surface is etched with a plasma to remove at least a portion of said surface.
13. The biocomposite according to any one of example embodiments 10 to 12, wherein the plasma is selected from the group comprising a cold argon-oxygen plasma, an oxygen plasma, a helium plasma, a nitrogen plasma, an argon plasma, or combinations thereof.
14. The biocomposite according to any one of the preceding example embodiments wherein the filler comprises at least one inorganic compound selected from: hydroxyapatite, silica (optionally silica particulates), glass fibres (optionally selected from E type and S type glass type fibres), bioactive glass fibres, and mixtures thereof.
15. The biocomposite according to any one of the preceding example embodiments wherein the filler comprises hydroxyapatite in a particulate form selected from nano-spheres, nano-whiskers and nano-rods.
16. The biocomposite according to any one of the preceding example embodiments wherein the filler comprises hydroxyapatite in a particulate form with a diameter of about 50 nm to about 200 nm.
17. The biocomposite according to any one of the preceding example embodiments wherein the filler comprises silica nano-particulates having a particle size of about 50 nm to about 700 nm.
18. The biocomposite according to any one of the preceding example embodiments, wherein the filler comprises glass fibres (optionally selected from E type and S type) having:
   a length of about 250 to about 350 μm and/or
   a diameter of about 5-10 μm.
19. The biocomposite according to any one of the preceding example embodiments, wherein the at least one monomer suitable for photopolymerisation comprises at least one monomer comprising at least one photopolymerisable: alkene, ally, vinyl, methacrylate and/or acrylate group, and mixtures thereof.
20. The biocomposite according to any one of the preceding example embodiments, wherein the at least one monomer suitable for photopolymerisation comprises at least one monomer comprising a plurality of photopolymerisable groups, optionally selected from: alkene, ally, vinyl, methacrylate and/or acrylate groups.
21. The biocomposite according to any one of the preceding example embodiments, wherein the at least one monomer suitable for photopolymerisation comprises: two or three methacrylate groups; two or three acrylate groups; two or three vinyl groups; or two or three allyl groups.
22. The biocomposite according to any one of the preceding example embodiments, wherein the at least one monomer suitable for photopolymerisation comprise at least one monomer selected from: urethane dimethacrylate, 2-hydroxyethyl methacrylate, triethylene glycol dimethacrylate, bisphenol A glycidyl methacrylate, ethoxylated bisphenol A dimethacrylate, decanediol dimethacrylate, and mixtures thereof.
23. The biocomposite according to any one of the preceding example embodiments, wherein the photoinitiator is selected from: camphorquinone, ethyl 4-dimethylaminobenzoate, trimethylbenzoyl-diphenyl-phosphine oxide, and mixtures thereof.
24. The biocomposite according to any one of the preceding example embodiments, wherein the one or more monomers are present in a range of about 70 to about 95 wt % of the biocomposite.
25. The biocomposite according to any one of the preceding example embodiments, wherein the filler composition is present in a range of about 5 to about 30 wt % of the biocomposite.
26. The biocomposite according to any one of the preceding example embodiments, further comprising at least one additive selected from: plasticiser, dye, pigment, modifier, stabiliser, acid scavenger, compatibiliser, other polymers, pharmaceutically active compound, or mixtures thereof.
27. The biocomposite according to any one of the preceding example embodiments, further comprising at least one additive which is a pharmaceutically active compound.
28. The biocomposite according to any one of the preceding example embodiments, wherein the biocomposite is substantially free or free of metal, optionally selected from titanium, iron, aluminium, magnesium, and copper, and alloys or mixtures thereof.
29. The biocomposite according to any one of the preceding example embodiments, wherein the biocomposite is substantially free or free of a polyaryletherketone, optionally selected from: polyether ether ketone (PEEK), polyether ketone (PEK), polyether ether ketone ketone (PEEKK), polyether ketone ketone (PEKK), or a mixture thereof.
30. The biocomposite according to any one of the preceding example embodiments, wherein the biocomposite is used for the treatment or repair of damaged or defective bone in a subject.
31. The biocomposite according to example embodiment 30, wherein the treatment or repair is a consequence of: trauma, optionally low or high energy trauma; tumour; and/or infection.
32. The biocomposite according to example embodiment 30 or example embodiment 31, wherein the treatment or repair is in the metaphysis, subchondral regions or diaphysis or any regions in flat bones or vertebrae.
33. The biocomposite according to any one of the preceding example embodiments, wherein the biocomposite is in the form of a medical device.
34. The biocomposite according to any one of the preceding example embodiments, wherein the biocomposite is in the form of an implant.
35. The biocomposite according to any one of the preceding example embodiments, wherein the biocomposite is in the form of an orthopaedic implant.
36. A method of synthesising a biocomposite according to any one of example embodiments 1 to 35, the method of comprising the steps of:
   providing a composition comprising:
      at least one monomer suitable for photopolymerisation
      a photoinitiator and
      a filler composition comprising at least one inorganic compound and
   3D printing the biocomposite.
37. The method according to example embodiment X, wherein the 3D printing is stereolithography 3D printing, masked stereolithography 3D printing, or digital light processing.
38. The method according to example embodiment 36 or example embodiment 37, wherein the 3D printing uses a ultraviolet light source.
39. The method according to example embodiment any one of example embodiments 36 to 38, wherein the 3D printing uses a light source with a wave length in the range of about 280 nm to about 400 nm.
40. The method according to any one of example embodiments 36 to 39, wherein:
   the composition further comprises at least one of: hydroxyapatite, n-hydroxyapatite, glass fibres and/or glass particles or
   the method further comprises the step of applying a layer comprising hydroxyapatite is applied to at least a portion of one or more surfaces of the biocomposite.
41. The method according to example embodiment 40, wherein the layer comprising hydroxyapatite is applied using a 3D printing technique.
42. The method according to any one of example embodiments 36 to 41, wherein at least one surface of the biocomposite is modified in a step comprising a process selected from: chemical etching, chemical coating (optionally chemical grafting), electrochemical grafting, laser etching, mechanical surface modifications, plasma-assisted coating, plasma-assisted etching, physical vapour deposition (optionally with a plasma or ion beam), chemical vapour deposition, atomic layer deposition, or mixtures thereof.
43. The method according to any one of example embodiments 36 to 42, wherein at least one surface of the biocomposite is etched to remove at least a portion of said surface, wherein the etching optionally comprises at least one technique selected from: chemical etching, laser etching, plasma-assisted etching, mechanical etching, or mixtures thereof.
44. The method according to example embodiment 43, wherein the etching is undertaken at a temperature in a range of about 20° C. to about 50° C., optionally in a range of about 25° C. to about 42° C.
45. The method according to any one of example embodiments 36 to 44, wherein at least one surface of the biocomposite is treated with a plasma.
46. The method according to example embodiment 45, wherein the treatment with a plasma increases the concentration of at least one of: hydroxyl groups, hydroxyl radicals and/or or reactive oxygen species, on at least a portion of the surface treated with the plasma.
47. The method according to any one of example embodiments 36 to 46, wherein at least one surface is etched with a plasma to remove at least a portion of said surface.
48. The method according to any one of example embodiments 36 to 47, wherein the plasma is selected from the group comprising a cold argon-oxygen plasma, an oxygen plasma, a helium plasma, a nitrogen plasma, an argon plasma, or combinations thereof.
49. A biocomposite formed from the method of any one of example embodiments 36 to 48.
50. A method of treating or repairing a damaged or defective bone in a subject, the method comprising administering biocomposite according to any one of example embodiments 1 to 35 to a subject in need thereof.
51. The method according to example embodiment 50, wherein the treatment or repair is a consequence of: trauma, optionally low or high energy trauma; tumour; and/or infection.
52. The method according to example embodiment 50 or example embodiment 51, wherein the treatment or repair is in the metaphysis, subchondral regions or diaphysis or any regions in flat bones or vertebrae.
53. Use of a composition comprising:
   at least one monomer suitable for photopolymerisation
   a photoinitiator and
   a filler composition,
      in the formation of a biocomposite according to any one of example embodiments 1 to 35.
54. Use of a biocomposite according to any one of example embodiments 1 to 35 in the formation of an article to treat or repair a bone in a subject.
55. The use according to example embodiment 54, wherein the treatment or repair is a consequence of: trauma, optionally low or high energy trauma; tumour; and/or infection.
56. The use according to example embodiment 54 or example embodiment 55, wherein the treatment or repair is in the metaphysis, subchondral regions or diaphysis or any regions in flat bones or vertebrae.
57. The use according to any one of example embodiments 54 to 56, wherein the article is a medical device.
58. The use according to any one of example embodiments 54 to 57, wherein the article is an implant.
59. The use according to any one of example embodiments 54 to 57, wherein the articles is an orthopaedic implant.

### EXAMPLES

The present disclosure will now be described with reference to the following non-limiting examples and with reference to the accompanying Figures.

### Example 1

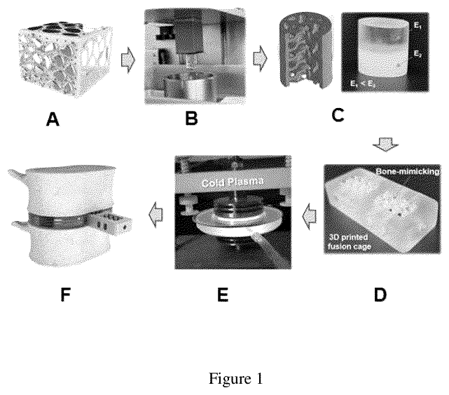

A development process for a multi-functionalised spinal fusion cage was designed and implemented using the stereolithography 3D printing and atmospheric cold plasma etching techniques, as shown in FIG. 1. A pure dental resin (PDR) and compositse were used in the preparation of the fusion cage (FIG. 2). To add mechanical, physical, and biological functions in the biocomposites, a nano-hydroxyapatite (n-HAP, spherical, D<200 nm) and strontium-doped glass particle (D<700 nm) were mixed as filler materials. n-HAP powder and its high magnification scanning electron microscope (SEM) image are presented in FIG. 3.

The PDR consisted of:
a mixture of 1,6-bis(methacryloxy-2-ethoxycarbonylamino)-2,4,4-trimethylhexane (UDMA, 80 wt. %) and triethylene glycol dimethacrylate (TEGDMA, 20 wt. %);
camphorquinone (CQ, 0.2 wt. %)-photoinitiator;
ethyl-4-dimethylamino benzoate (EDMAB, 0.5 wt. %)-photoinitiator; and
butylated hydroxytoluene (BHT, 0.05 wt. %)-inhibitor.

The composite groups consisted of:
GP10-PDR (90 wt. %) and strontium glass particle (10 wt. %);
GP10+nHAp02-PDR (88%), strontium glass particle (10 wt. %), and n-HAP (2 wt. %);
GP20−PDR (80 wt. %) and strontium glass particle (20 wt. %);
GP20+nHAp02-PDR (78%), strontium glass particle (20 wt. %), and n-HAP (2 wt. %);

The spinal fusion cage (FIG. 2—image 1) was designed with multi-layers (FIG. 2—image 2) on the top and bottom of the cage to improve an osteoinductive property. Also, a repetitive microporous structure (FIG. 2—image 3) was integrated at the middle of the cage to improve osteoconductive capability. The main body of the cage was 3D printed using PDR and the functionalized top/bottom layers were printed using the GP20+n-HAP composite. The 3D printed cage was rinsed in an ultrasonic bath with isopropyl alcohol and post-cured under the UV and blue lights at 60° C. for 1 hour.

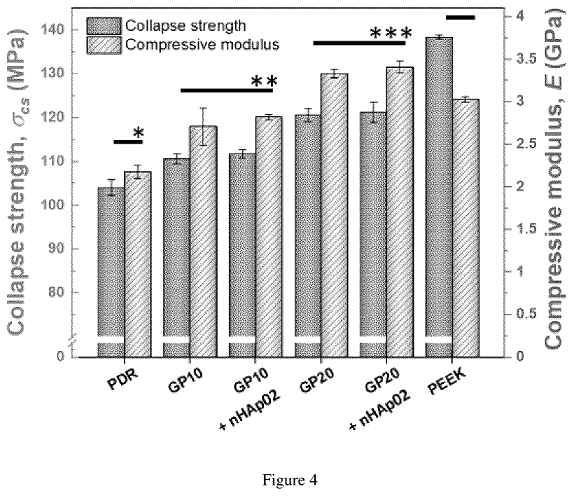

To evaluate the effect of fillers on the mechanical properties, compressive collapse strength and modulus of the printed specimens in each group were measured with a compression test. The circular pillar specimens (length: 10 mm, diameter: 7 mm), were prepared using 3D printing and all 3D printed specimens were post-cured before the tests. PDR was used as a control and composite specimens were used as an experimental group. Also, polyether ether ketone (PEEK) was tested for comparison. FIG. 4 shows the compressive collapse strength and modulus, where a statistically significant improvement in both properties is observed as increasing the filler loading in the composites compared to PDR. As compared to the measured values of PEEK, the collapse strength of the composites is lower, but the modulus shows higher than PEEK. All composites of the present example exhibited higher compressive properties than the control, and two groups, GP20 and GP20+nHAp02, exhibited higher collapse strength than PEEK, indicating the good suitability of the biocomposites of the present disclosure for use in high compression stress areas.

To improve the surface performance of the biocomposite for bone regeneration and adhesion, atmospheric cold plasma was applied on the 3D printed implant structures. The plasma was performed in a dielectric barrier discharge plasma reactor and atmospheric pressure. The temperature in the reactor during the process was maintained between 23-35° C. Argon and oxygen gas mixture (9:1) was used as a processing gas and was introduced directly to the discharge area via the side gas tube. The RF power was set at 50 W, and specimens were treated for up to 4 minutes total plasma on-time. The surface observation of the plasma etched composites was performed by means of SEM and hydrophilic properties of the treated surface were examined by water contact angle measurements as shown in FIG. 5.

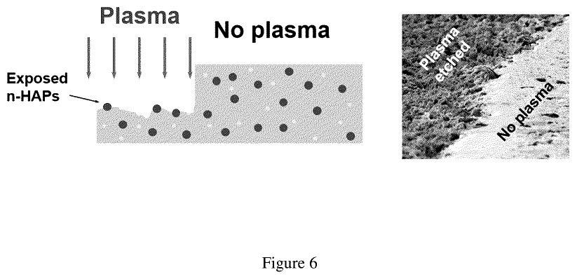

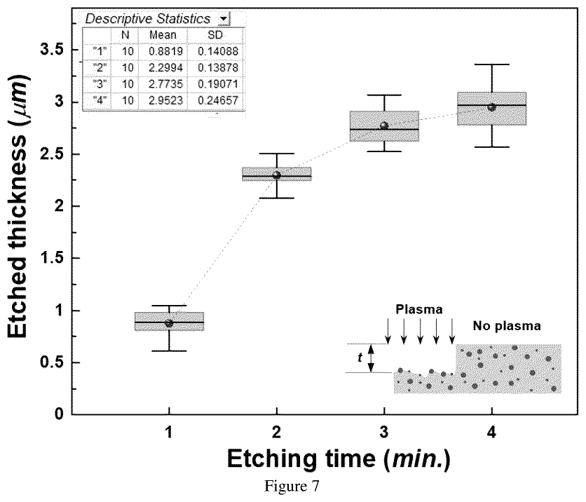

The phenomenon of surface plasma etching or texturing is related to different etching rates among different materials in the composites, and a high etching selectivity can be achieved in a combination of different phases of materials such as polymer composites reinforced with inorganic filler materials. As increasing plasma treatment time, the polymer matrix is highly etched and inorganic fillers such as glass particles and n-HAPs would be remained and exposed on the outer surface of the composites as shown in FIG. 6. The surface roughness increased in nanoscale. Also, oxygen atoms create hydroxyl groups (—OH groups) on the surface of the exposed filler materials and they change the surface characteristics of the composites from hydrophobicity to hydrophilicity with the increase of surface energy. As a result, the water contact angle, a measure of wettability, decreased from 115 to 50 degrees after 2 minutes of plasma treatment (FIG. 5). The etching rate of the composite (GP20+nHAp02) under the argon-oxygen plasma was calculated as shown in FIG. 7.

Based on the experimental results, it is assumed that the disclosed composites with the proposed surface modification process would enhance the cell attachment and proliferation on the 3D printed fusion cages and implants.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

1. A biocomposite formed from a composition comprising:
at least one monomer suitable for photopolymerisation
a photoinitiator and
a filler composition comprising at least one inorganic compound.

2. The biocomposite according to claim 1, wherein:
at least one surface of the biocomposite is modified in a process selected from: chemical etching; chemical coating; chemical grafting; electrochemical grafting; laser etching; mechanical surface modifications; plasma-assisted coating; plasma-assisted etching; physical vapour deposition, physical vapour optionally with a plasma or ion beam; chemical vapour deposition; atomic layer deposition; or mixtures thereof; or
at least one surface of the biocomposite is etched to remove at least a portion of said surface; or
at least one surface of the biocomposite is treated with a plasma.

3-5. (canceled)

6. The biocomposite according to claim 2, wherein the treatment with a plasma increases the concentration of at least one of: hydroxyl groups, hydroxyl radicals, and reactive oxygen species, on at least a portion of the surface treated with the plasma; or
the plasma is selected from a cold argon-oxygen plasma, an oxygen plasma, a helium plasma, a nitrogen plasma, an argon plasma, and combinations thereof.

7-9. (canceled)

10. The biocomposite according to claim 1, wherein the biocomposite is:
formed via 3D printing using a ultraviolet light source; or
formed via 3D printing using a light source with a wavelength in the range of about 280 nm to about 400 nm.

11. (canceled)

12. The biocomposite according to claim 1, wherein:
the biocomposite further comprises at least one of: hydroxyapatite, n-hydroxyapatite, glass fibres or glass particles or
a layer comprising: hydroxyapatite, n-hydroxyapatite, glass fibres glass particles is applied to at least a portion of one or more surfaces of the biocomposite.

13. (canceled)

14. The biocomposite according to claim 1, wherein the filler comprises:
at least one inorganic compound selected from: hydroxyapatite; silica; glass fibres, E type glass fibres, S type glass fibres, bioactive glass fibres, and mixtures thereof; or
hydroxyapatite in a nano-spheres, nano-whiskers and nano-rods;
hydroxyapatite in a particulate form with a diameter of 50 nm to 200 nm; or
silica nano-particulates having a particle size of 50 nm to 700 nm.

15-18. (canceled)

19. The biocomposite according to claim 1, wherein the at least one monomer suitable for photopolymerisation comprises:
at least one monomer comprising at least one photopolymerisable: alkene, ally, vinyl, methacrylate or acrylate group, and mixtures thereof; or
at least one monomer comprising a plurality of photopolymerisable groups, selected from: alkene, ally, vinyl, methacrylate or acrylate groups; or
two or three methacrylate groups; two or three acrylate groups; two or three vinyl groups; or two or three allyl groups; or
at least one monomer selected from: urethane dimethacrylate, 2-hydroxyethyl methacrylate, triethylene glycol dimethacrylate, bisphenol A glycidyl methacrylate, ethoxylated bisphenol A dimethacrylate, decanediol dimethacrylate, and mixtures thereof.

20-26. (canceled)

27. The biocomposite according to claim 1, further comprising at least one additive which is a pharmaceutically active compound.

28-32. (canceled)

33. The biocomposite according to claim 1, wherein the biocomposite is in the form of a medical device, an implant, or an orthopaedic implant.

34-35. (canceled)

36. A method of synthesising a biocomposite comprising:
providing a composition comprising:
at least one monomer suitable for photopolymerisation
a photoinitiator and
a filler composition comprising at least one inorganic compound and
3D printing the biocomposite.

37. The method according to claim 36, wherein the 3D printing is stereolithography 3D printing, masked stereolithography 3D printing, or digital light processing; or an ultraviolet light source; or uses a light source with a wave length in the range of 280 to 400 nm.

38-39. (canceled)

40. The method according to claim 36, wherein:
the composition further comprises at least one of: hydroxyapatite, n-hydroxyapatite, glass fibres or glass particles; or
the method further comprises applying a layer comprising hydroxyapatite is applied to at least a portion of one or more surfaces of the biocomposite.

41. The method according to claim 40, wherein the layer comprising hydroxyapatite is applied using a 3D printing technique.

42. The method according to claim 36, wherein:
at least one surface of the biocomposite is modified via a process_selected from: chemical etching; chemical coating; chemical grafting; electrochemical grafting; laser etching; mechanical surface modifications; plasma-assisted coating; plasma-assisted etching; physical vapour deposition, physical vapour deposition with a plasma or ion beam; chemical vapour deposition; atomic layer deposition; or mixtures thereof; or
at least one surface of the biocomposite is etched to remove at least a portion of said surface; or
at least one surface of the biocomposite is etched to remove at least a portion of said surface using at least one technique selected from: chemical etching, laser etching, plasma-assisted etching, mechanical etching, or mixtures thereof.

43-44. (canceled)

45. The method according to claim 36, wherein at least one surface of the biocomposite is treated with a plasma.

46. The method according to claim 45, wherein the treatment with a plasma increases the concentration of at least one of: hydroxyl groups, hydroxyl radicals, and reactive oxygen species, on at least a portion of the surface treated with the plasma.

47. The method according to claim 36, wherein at least one surface of the biocomposite is etched with a plasma to remove at least a portion of said surface.

48. The method according to claim 36, wherein the plasma is selected from the group comprising a cold argon-oxygen plasma, an oxygen plasma, a helium plasma, a nitrogen plasma, an argon plasma, or combinations thereof.

49. (canceled)

50. A method of treating or repairing a damaged or defective bone in a subject, the method comprising administering to a subject in need thereof a biocomposite comprising:
at least one monomer suitable for photopolymerisation,
a photoinitiator, and
a filler composition comprising at least one inorganic compound.

51. The method according to claim 50, wherein the treatment or repair is a consequence of: trauma, low energy trauma, high energy trauma; tumour; infection; or in the metaphysis, subchondral regions, or diaphysis or any regions in flat bones or vertebrae.

52-59. (canceled)
